(19) United States
(12) Patent Application Publication
(10) Pub. No.: US 2015/0239956 A1
(43) Pub. Date: Aug. 27, 2015

(54) HIGH-AFFINITY ANTIBODY AND METHOD FOR MANUFACTURING THE SAME

(71) Applicants: ASAHI KASEI MEDICAL CO., LTD., Tokyo (JP); NOMADIC BIOSCIENCE CO., LTD., Okayama (JP)

(72) Inventors: Ichiro Koguma, Tokyo (JP); Kazuo Okuyama, Tokyo (JP); Satoshi Sato, Okayama (JP)

(73) Assignees: ASAHI KASEI MEDICAL CO., LTD., Tokyo (JP); NOMADIC BIOSCIENCE CO., LTD., Okayama (JP)

(21) Appl. No.: 14/408,458

(22) PCT Filed: Jun. 27, 2013
(86) PCT No.: PCT/JP2013/067705

§ 371 Date: Dec. 16, 2014

(30) Foreign Application Priority Data
Jun. 27, 2012 (JP) 2012-144742

(51) Int. Cl. C07K 16/00

(52) U.S. Cl. CPC C07K 16/00; C07K 2317/21

(57) ABSTRACT

A method for manufacturing a high-affinity antibody including purifying an antibody to which a treatment of reducing the affinity for an antigen is not applied by a temperature responsive protein A medium, in which dissociation constant (K_{D} value) to an antigen is smaller than the K_{D} value of an antibody purified by an acid elution type protein A medium.

### TECHNICAL FIELD

The present invention relates to a purification technique, and particularly to an antibody purification method.

### BACKGROUND ART

Immunoglobulins (antibody) are physiologically active substances responsible for immunoreactions. Recently, availability of antibodies in the use of e.g., medicinal products, diagnostic drugs and materials for separating/purifying the corresponding antigen proteins has been increased. Antibodies can be obtained from e.g., blood of immunized animals, culture solutions of cells having antibody productivity, and culture solutions of ascitic fluids taken from animals. However, the blood and culture solutions contain impurities such as proteins other than antibodies and complicated admixtures. Thus, in order to purify an antibody by separating it from impurities, usually, a complicated and time-consuming purification step is required.

As a method for manufacturing a high-purity immunoglobulin by removing impurities, affinity chromatography is mainly used as a technique.

In the affinity chromatography, an antibody having high purity and concentration can be purified through the following (A) to (C) steps:

(A) a step of loading a sample contaminated with impurities in a column (loading step)

(B) a step of removing impurities except an antibody as a target to be purified from the loaded column (washing step),

(C) a step of collecting the antibody as the target to be purified from the column (elution step).

In the loading step and washing step herein, the environment in the column is controlled such that the antibody as the target to be purified can strongly bind to an affinity ligand, which is bound to an immobilization phase of the chromatography; whereas, in the elution step, the environment of the column must be changed such that both ligand and antibody separate from each other. The environment change is usually made by pH change.

As the ligand of affinity chromatography for use in antibody purification, protein A derived from Staphylococcus and its antibody-binding domain, which has extremely high specificity and affinity for a common region of antibodies, are known and widely used in industrial-scale production steps for antibodies. Protein A generally binds to an antibody under physiological conditions and liberates the antibody under acidic conditions.

It has been recently proposed to use protein A capable of changing interaction with a biological material as a target to be isolated by changing temperature (hereinafter referred to as temperature responsive protein A) in affinity chromatography. Note that protein A, which has properties of binding to an antibody under physiological conditions and liberating the antibody under acidic conditions but incapable of changing the interaction with a biological material as a target to be isolated even if temperature is changed, will be hereinafter referred to as “acid elution type protein A”.

Patent Literature 1 discloses a method for purifying an antibody which is obtained by a commercially available means by using temperature responsive protein A and in the method for purifying an antibody disclosed in Example 8, human IgG obtained by a commercially available means is allowed to once adsorb to temperature responsive protein A and elute by changing temperature.

However, the antibodies that can be obtained by a commercially available means are generally subjected to a treatment, which reduces affinity for antigens in view of safety of viruses. For example, in the case where an antibody is a human monoclonal antibody derived from a recombinant cell, a low pH treatment (generally, pH3.0 to 4.0, one hour or more) is generally applied. However, in the antibody treated in low pH, inherent affinity of the antibody for an antigen is already damaged. It has been found that if the low pH treatment is further applied, the antibody is sterically changed and association or aggregation takes place, causing malfunction (for example, see Patent Literature 2). In the case where the antibody is a human polyclonal antibody derived from an animal, such as human blood plasma, a heat treatment (generally, 60° C., 10 hours) is generally applied (for example, see Patent Literature 3). Also in the antibody thus treated in a high temperature, its inherent affinity for an antigen is already damaged.

A method by which an antibody can be purified with high purity and in high yield while keeping high-affinity for an antigen without damaging the inherent affinity of the antibody for the antigen is industrially useful; however, such a method had not been found and studied.

### CITATION LIST

### Patent Literature

Patent Literature 1: International Publication No. WO08/143,199
Patent Literature 2: Japanese Patent Laid-Open No. 2005-206602
Patent Literature 3: Japanese Patent Laid-Open No. 2008-94722

### SUMMARY OF INVENTION

### Technical Problem

An object of the present invention is to provide a method for manufacturing an antibody maintaining the affinity for an antigen with high purity and in high yield.

### Solution to Problem

At present, a human monoclonal antibody as a biotechnology-based medicinal product is purified generally by an acid elution type protein A medium. However, as a result of intensive studies conducted by the present inventors, we found that an antibody having high-affinity for an antigen can be obtained with high purity and in high yield by purifying an antibody to which a treatment of reducing the affinity for an antigen is not applied by a temperature responsive protein A medium.

More specifically, the present invention provides a method for purifying an antibody to which a treatment of reducing the affinity for an antigen is not applied with high purity and in high yield and while maintaining the high-affinity for the antigen, by temperature responsive protein A, and provides a high-affinity antibody obtained thereby.

An aspect of the present invention provides a method for manufacturing a high-affinity antibody including: (A) a step of culturing a cell producing a monoclonal antibody, (B) a step of removing the cell from a solution containing the cell, and (C) a step of purifying the monoclonal antibody contained in the solution by a temperature responsive protein A medium, in which, before the step (C), none of a low pH treatment, a high-temperature treatment performed at 60° C. or more and a purification treatment by affinity chromatography, are included, and the solution obtained in the step (B) from which the cell is removed is subjected to the step (C) within 24 hours.

The dissociation constant (K_{D} value) of the obtained antibody to the antigen herein is smaller than the K_{D} value of the antibody obtained by a method for manufacturing an antibody including a purification step using an acid elution type protein A medium, which adsorbs a monoclonal antibody at pH4 to 9 and elutes the monoclonal antibody at pH2 to 4, in place of the step (C).

The dissociation rate constant (K_{d} value) of the obtained antibody to the antigen is smaller than the K_{d} value of the antibody obtained by a method for manufacturing an antibody including a purification step using an acid elution type protein A medium, which binds a monoclonal antibody at pH4 to 9 and elutes the monoclonal antibody at pH2 to 4, in place of the step (C).

The cell is, for example, a Chinese hamster ovary cell (CHO cell). The antibody is, for example, a human antibody.

It is preferable that after the antibody is purified by a temperature responsive protein A medium in the step (C), none of a low pH treatment, a high-temperature treatment performed at 60° C. or more and a purification treatment by affinity chromatography are performed.

Another aspect of the present invention provides a high-affinity antibody obtained by a method for manufacturing an antibody including: (A) a step of culturing a cell producing a monoclonal antibody, (B) a step of removing the cell from a solution containing the cell, and (C) a step of purifying the monoclonal antibody contained in the solution by a temperature responsive protein A medium, in which, before the step (C), none of a low pH treatment, a high-temperature treatment performed at 60° C. or more and a purification treatment by affinity chromatography, are included, and the solution obtained in the step (B) from which the cell is removed is subjected to the step (C) within 24 hours.

The dissociation constant (K_{D} value) of the high-affinity antibody to the antigen herein is smaller than the K_{D} value of the antibody obtained by a method for manufacturing an antibody including a purification step using an acid elution type protein A medium, which binds a monoclonal antibody at pH4 to 9 and elutes the monoclonal antibody at pH2 to 4, in place of the step (C).

The dissociation rate constant (K_{d} value) of the high-affinity antibody to the antigen is smaller than the K_{d} value of the antibody obtained by a method for manufacturing an antibody including a purification step using an acid elution type protein A medium, which binds a monoclonal antibody at pH4 to 9 and elutes the monoclonal antibody at pH2 to 4, in place of the step (C).

The cell is, for example, a Chinese hamster ovary cell (CHO cell). The antibody is, for example, a human antibody.

It is preferable that after the antibody is purified by a temperature responsive protein A medium in the step (C), none of a low pH treatment, a high-temperature treatment performed at 60° C. or more and a purification treatment by affinity chromatography are performed.

### Advantageous Effects of Invention

According to the present invention, it is possible to provide a method for purifying an antibody while maintaining high-affinity for an antigen, with high purity and in high yield, and the antibody obtained by the purification method.

### BRIEF DESCRIPTION OF DRAWINGS

FIG. 1 is a table showing the measurement results of affinity of antibodies in Examples and Comparative Examples.

### DESCRIPTION OF EMBODIMENTS

Embodiments of the present invention (hereinafter referred to simply as the “embodiment”) will be more specifically described. A method for manufacturing (purifying) a high-affinity antibody according to the embodiment includes purifying an antibody to which a treatment of reducing the affinity for an antigen is not applied by a temperature responsive protein A medium. The dissociation constant (K_{D} value) of the purified antibody to the antigen herein is smaller than the K_{D} value of the antibody purified by an acid elution type protein A medium.

The antibody described in the embodiment refers to a glycoprotein molecule (also refers to gamma globulin or immunoglobulin) produced by B lymphocytes serving as a vertebrate infection defense mechanism, as generally defined in biochemistry. Particularly, antibodies that can be used as a medicinal product to a human are very useful as a medicinal product and have a property that the affinity of the antibodies for an antigen tends to easily reduce. For these reasons, the antibodies are suitably produced according to the embodiment. More specifically, the antibodies that can be used as a medicinal product to a human, are virtually not allowed to mix with pathogenic microorganisms such as viruses, and have substantially the same structure as those of antibodies present in the human body as a subject to be administered.

The type of antibody, more specifically, class (isotype) and subclass of the antibody, are not particularly limited. For example, antibodies are classified into 5 classes such as IgG, IgA, IgM, IgD and IgE depending upon the difference in structure of the constant region. Any type of immunoglobulin can be used. In human antibodies, IgGs have four subclasses of IgG1 to IgG4; however, any IgG can be used. Particularly, IgG1 and IgG4, which are highly useful as an antibody medicament, are suitably produced by the method according to the embodiment since their affinity for an antigen significantly tends to reduce. IgAs have two subclasses of IgA1 and IgA2, which are not particularly limited, either. Note that as long as it is applicable as a medicinal product, an antibody-associated protein with a bound Fc region also falls within the range of the antibody according to the embodiment.

In the embodiment, a chimeric antibody with a human IgG refers to an antibody having variable regions derived from an organism except a human, such as a mouse, and the other regions, i.e., constant regions, substituted by human immunoglobulins. Furthermore, a humanized antibody refers to an antibody which has the complementarity-determining regions (CDR) of variable regions derived from an organism except a human and the other regions, i.e., framework regions (FR), derived from a human. The immunogenicity of the humanized antibody is further lower than that of the chimeric antibody.

Antibodies can be also classified based on the origins and production methods. Either one of a naturally occurring human antibody and a recombinant human antibody produced by a gene recombination technique or either one of a monoclonal antibody and a polyclonal antibody can be used. Among these antibodies, application to a human IgG is preferable in view of demands and importance as an antibody medicament. Furthermore, the antibody purification conditions of the embodiment including a specific ligand and a specific pH described later are favorable for purification of a human IgG.

The antibody serving as a medicinal product is produced through the following summarized steps: a cell culture step, a cell separation (removal) step, a purification step, a virus removal step, concentration/liquid exchange step and a bottling step in this order. Needless to say, the flowchart of production is not limited to this. An additional step may be added or individual steps may be partly exchanged. The above flowchart is a typical one for manufacturing a desired antibody by a cell culture method. In the case where a desired antibody is purified from a human body fluid or a cell culture solution, the cell culture step and the cell separation step are skipped and the body fluid or the cell culture solution is added to the purification step.

The solution to be added in the purification step contains the antibody to be purified and impurities. The impurities are removed from the solution by the purification step to purify the antibody. The impurities may contain admixtures, proteins other than the antibody to be purified and antibody aggregates. The antibody aggregates are formed of, for example, multimers (dimer or more) of antibodies. In the case where the solution to be added in the purification step is a body fluid, examples of the body fluid include blood, plasma, serum, lymph fluid, ascitic fluid, hydrothorax, or a solution mixture thereof, a dilution thereof prepared by adding a physiological solution such as physiological saline, buffer and sterile water to them, and a blood preparation.

In the case where the solution to be added in the purification step is a cell culture solution, the cell culture solution can be obtained by removing cells from a cell suspension solution by filtration and precipitation. The cell culture solution may be diluted with a physiological solution. The cell culture solution contains antibodies released outside the cells or secreted from the cells during culture. Examples of the cell suspension solution may include a solution in which cells cultured in order to obtain a medicament raw-material solution are suspended. Examples of the cells include cells taken from animal body-fluids and tissues, established cell lines that are artificially concerted, and these cells cultured outside a living body.

In the embodiment, the antibody before purification by a temperature responsive protein A medium is an antibody which is not subjected to a treatment of substantially reducing the affinity for an antigen. Examples of the treatment of reducing the affinity for an antigen include, but not particularly limited to, a virus inactivation treatment or a purification treatment by affinity chromatography. Examples of the virus inactivation treatment include a low pH treatment, a high-temperature treatment performed at 60° C. or more, treatment by UV irradiation, a treatment by pigment addition and a treatment by a solvent detergent method; however, a low pH treatment and a high-temperature treatment performed at 60° C. or more are the most common. Furthermore, examples of the purification treatment by affinity chromatography include a purification treatment with acid elution type protein A.

In the embodiment, it is desirable to substantially lower as much as possible a risk of reducing the affinity for an antigen in a collection step of the antibody before purification by a temperature responsive protein A medium. Examples of the risk of reducing the affinity for an antigen in the collection step include time interval required from the cell separation step to the purification step. In the case where Chinese hamster ovary cells (CHO cells), which are most generally used in manufacturing antibodies, are used in the cell culture step, the culture supernatant after the cell separation step is contaminated with impurities derived from the CHO cells, such as a proteolytic enzyme. If these impurities are in contact with an antibody for a long time, the affinity of the antibody for an antigen decreases. To lower the risk of reducing the affinity for an antigen, time interval required from a cell separation (removal) step in which cells are removed from a solution containing the cells, to an antibody purification step by a temperature responsive protein A medium is controlled to fall within preferably 24 hours, more preferably 12 hours and further preferably 6 hours.

In the method according to the embodiment, a purification treatment with acid elution type protein A is not included at all. In the purification treatment with acid elution type protein A, to elute an antibody from a column by changing pH, hydrogen ion exponent (pH) is changed from a neutral region (the pH of the loading/washing step) of pH6 to 8, in which the affinity of an antibody for protein A is high, to an acidic region (the pH of the elution step) of pH3 to 4, in which the affinity extremely reduces. In the acidic pH region, the steric structure of an antibody changes and association and aggregation occur, with the result that malfunction of the antibody takes places.

In the method according to the embodiment, any virus inactivation treatment, which substantially reduces the affinity for an antigen, is not included at all. In the virus inactivation treatment, an eluate from acid elution type protein A is generally incubated at the same low pH (pH3 to 4) at room temperature for about one hour. After a lapse of a predetermined time, a buffer such as an aqueous sodium hydroxide solution and Tris/hydrochloric acid buffer of about pH5 to 9 and a phosphate buffer, is added dropwise to increase the hydrogen ion exponent (pH) to about pH5 to 7, i.e., neutralize the eluate.

In the purification step according to the embodiment, temperature responsive protein A is used. The medium for use in temperature responsive protein A has a support and a temperature responsive ligand, whose affinity for an antibody as a target to be purified) changes in response to the temperature and which is introduced in the surface of the support.

Examples of the form of the support that the medium has include, but not particularly limited to, film form such as a flat film and a hollow fiber or bead-form. The hollow fiber support is favorably used since it is easy to mold a module and the area of the film per module is large. A bead support is also favorably used since the surface area per volume is generally larger compared to the surface area per volume of film support, and a large amount of antibody can be adsorbed.

The material for the support is not particularly limited. In the case where the support is a film, a polymer material capable of forming a porous film is favorably used. Examples of the material that can be used for the support include olefin resins such as polyethylene and polypropylene, polyester resins such as polyethylene terephthalate and polyethylene terenaphthalate, polyamide resins such as nylon 6 and nylon 66, fluorine-containing resins such as polyvinylidene fluoride and polychlorotrifluoroethylene, and amorphous resins such as polystyrene, polysulfone, polyethersulfone and polycarbonate.

In the case where the support is a bead, glass, silica, a polystyrene resin, a methacrylic resin, crosslinked agarose, crosslinked dextran, a crosslinked polyvinyl alcohol and a crosslinked cellulose can be used as the material for the support. The crosslinked polyvinyl alcohol and crosslinked cellulose, since they are highly hydrophilic and can suppress adsorption of impurities, can be favorably used as the material for the support.

The support may have e.g., a plurality of micropores. The diameter of micropores is, but not particularly limited to, for example, 5 to 1000 nm, preferably 10 to 700 nm and further preferably 20 to 500 nm. If the diameter of the micropores is 5 nm or less, the molecular weight of the antibody to be separated tends to be low. In contrast, if the diameter of the micropores is 1000 nm or more, the surface area of the substrate decreases and the binding amount of antibody tends to be low.

Any coupling group may be introduced into the support. Examples of the coupling group include a carboxyl group activated by N-hydroxy succinimide (NHS), a carboxyl group, a cyanogen bromide activating group, a hydroxyl group, an epoxy group, an aldehyde group and a thiol group. In the case where the ligand to be introduced into a medium surface is temperature responsive protein A, the temperature responsive protein A has a primary amino group. Thus, among the coupling groups mentioned above, e.g., a carboxyl group activated by NHS, a carboxyl group, a cyanogen bromide active group, an epoxy group and a formyl group, which are capable of coupling with a primary amino group, are favorably used. For example, the carboxyl group activated by NHS is advantageous since other chemical agents are not required during a coupling reaction, the reaction quickly proceeds and a strong bond is formed with a primary amino group.

A spacer may be inserted between the support and the coupling group. A method of introducing a coupling group into a support is disclosed in various literatures.

A graft polymer chain having the coupling group at an end and/or a side chain may be introduced into the support. The density of the coupling group can be controlled or optionally improved by introducing a graft polymer chain having the coupling group into the support. A polymer chain having the coupling group may be grafted to the support or a polymer chain having a precursor functional group that can be converted into the coupling group may be grafted to the support and thereafter, the grafted precursor functional group may be converted into the coupling group.

As a method for introducing the graft polymer chain, any method may be employed. For example, the polymer chain is previously prepared and may be coupled with the support. Alternatively, the graft chain may be directly polymerized on the support by a “living radical polymerization method” or a “radiation graft polymerization method”. The “radiation graft method” is favorably used since it is not necessary to previously introduce a reaction initiator into the support and a wide variety of supports are applicable.

When the graft chain is introduced by a “radiation graft polymerization method”, any means may be employed for generating radicals from the support. However, in order to uniformly generate radicals from the entire support, it is preferable to irradiate withionizing radiation. The types of ionizing radiation that can be used include a gamma beam, an electron beam, a beta beam and a neutron beam. In consideration of use on an industrial scale, the electron beam or the gamma beam is preferable. Ionizing radiation can be obtained from a radioactive isotope such as cobalt-60, strontium 90 and cesium-137 or by e.g., an X-ray equipment, an electron beam accelerator and an ultraviolet lamp.

The irradiation dose of ionizing radiation is, for example, preferably 1 kGy or more and 1000 kGy or less, more preferably 2 kGy or more and 500 kGy or less, and further preferably 5 kGy or more and 200 kGy or less. If the irradiation dose is less than 1 kGy, radicals are unlikely to be uniformly generated. In contrast, if the irradiation dose exceeds 1000 kGy, the physical strength of a support is likely to decrease.

Graft polymerization methods by irradiation of ionizing radiation are generally classified into a pre-irradiation method, in which radicals are allowed to generate from a support and then brought into contact with a reactive compound, and a simultaneous irradiation method, in which radicals are generated from a support while the support is allowed to be in contact with a reactive compound. In the embodiment, any method can be applied; however, the pre-irradiation method is preferable since generation of oligomers is low.

The solvent to be used in graft polymerization is not particularly limited as long as it can homogeneously dissolve the reactive compound. Examples of such a solvent include an alcohol such as ethanol, isopropanol and t-butyl alcohol; an ether such as diethyl ether and tetrahydrofuran; a ketone such as acetone and 2-butanone; water, or a mixture thereof.

Examples of the monomer having the coupling group for use in graft polymerization include monomers such as acrylic acid and methacrylic acid in the case where the carboxyl group is used as the coupling group. The examples include an allylamine in the case where the primary amino group is used as the coupling group. The examples include glycidyl methacrylate (GMA) in the case where the epoxy group is used as the coupling group. Glycidyl methacrylate is industrially favorably used because it can form various functional groups by use of ring opening reactions of various epoxy groups.

In the case where the carboxyl group is used as the coupling group, glycidyl methacrylate is firstly subjected to graft polymerization and then the epoxy group of glycidyl methacrylate is hydrolyzed to obtain a diol. Subsequently, a hydroxy group derived from the diol is subjected to a ring-opening half esterification reaction with a cyclic acid anhydride to form the carboxyl group derived from the cyclic acid anhydride (ring-opening half esterification reaction). In view of production cost, the cyclic acid anhydride is desirably succinic anhydride or glutaric anhydride, but not limited to these.

The catalyst to be used in the ring-opening half esterification reaction is not particularly limited as long as it accelerates the reaction. Specific examples thereof include triethylamine, isobutylethylamine, pyridine and 4-dimethylaminopyridine. Among these, triethylamine or 4-dimethylaminopyridine is preferable. In view of the reaction rate and yield, 4-dimethylaminopyridine is the most preferable.

The ring-opening half esterification reaction is preferably performed in an inactive organic solvent such as toluene to which the above catalyst is added.

The carboxyl group formed by the ring-opening half esterification reaction is converted into an active ester by the activation reaction by NHS. Compared to the carboxyl group, the reactivity of the active ester is higher. Because of this, if it is desired to quickly immobilize a ligand such as temperature responsive protein A on the medium, the active esterification step is preferably performed.

The active ester has a chemical structure of R—C(═O)—X. X represents a leaving group such as a halogen, N-hydroxysuccinimide group or a derivative thereof, 1-hydroxybenzotriazole group or a derivative thereof, a pentafluorophenyl group and a para-nitrophenyl group, but not limited to these. As the active ester, N-hydroxysuccinimide ester is desirable in view of reactiveness, safety and production cost. The carboxyl group is converted into N-hydroxysuccinimide ester by simultaneously reacting a N-hydroxysuccinimide and a carbodiimide with the carboxyl group.

The carbodiimide is an organic compound having a chemical structure of —N═C═N—. Examples of the carbodiimide include, but not limited to, dicyclohexylcarbodiimide, diisopropylcarbodiimide and 1-ethyl-3-(3-dimethylaminopropyl)carbodiimide hydrochloride. The concentrations of the N-hydroxysuccinimide and carbodiimide are desirably set within the range of 1 to 100 mmol/L, and the reaction temperature within the range of 0° C. or more and less than 100° C., and the reaction time within the range of 2 minutes to 16 hours. As the reaction solvent, e.g., N,N′-dimethylformamide (DMF) and toluene can be used.

To a substrate for the aforementioned temperature responsive affinity chromatographic medium, temperature responsive protein A is introduced as the temperature responsive ligand. Temperature responsive protein A is protein A modified so as to change the affinity for an antibody depending upon the temperature. Specifically, temperature responsive protein A binds to an antibody at a low temperature and dissociates from the antibody at a higher temperature than the temperature at which it binds to an antigen. Temperature responsive protein A can be prepared with reference to Patent Literature (WO2008/143199).

The coupling reaction between the NHS-activated carboxyl group and temperature responsive protein A is, for example, performed as follows. First, a solution containing temperature responsive protein A in a concentration of 0.1 to 100 mg/mL is prepared by using a buffer containing no amino-group component, such as a citric acid buffer (pH3.0 to 6.2), an acetic acid buffer (pH3.6 to 5.6), a phosphate-buffered saline (PBS, pH5.8 to 8.5) or a carbonic acid buffer (pH9.2 to 10.6). When the aqueous solution is brought into contact with the active ester on the medium surface, the functional group such as the amino group contained in temperature responsive protein A reacts with the active ester to form the amide bond. As a result, temperature responsive protein A is covalently immobilized to the active ester on the medium surface. The contact time herein may be set within the range of 2 minutes to 16 hours. After temperature responsive protein A is immobilized to the medium surface, the medium is desirably washed with an appropriate washing solution. The washing solution herein is desirably a buffer containing about 0.5 mol/L of salt (NaCl) and about 0.1% nonionic surfactant. Owing to this, temperature responsive protein A, which only physically adsorbs without covalently binding, can be removed from the medium surface.

After temperature responsive protein A is immobilized to the medium surface (preferably after the medium having temperature responsive protein A immobilized thereto is further washed), it is preferable to bind an unreacted carboxyl group or an active ester to a low molecular compound having an amino group to protect the unreacted carboxyl group or the active ester. In this manner, molecules other than a purification subject, such as impurities, can be prevented from undesirably immobilizing to the medium surface. In particular, in the case where the functional group introduced into the substrate surface of medium is the active ester, it is preferable to perform this operation.

In the specification, an operation of reacting the low molecular compound having the amino group with the active ester group is sometimes referred to as “blocking”. Note that the medium surface after the carboxyl group or the active ester is reacted with the low molecular compound is desirably hydrophilic. This is because a hydrophilic surface generally suppresses unspecific adsorption of biological materials to a substrate surface. For this, as the low molecular compound containing the amino group, a low molecular compound further having a hydrophilic group other than an amino group is preferably used. Examples of such a low molecular compound include, but not limited to, ethanolamine, tris(hydroxymethyl)aminomethane and diglycolamine (IUPAC name:2-(2-aminoethoxy)ethanol). These low molecular compounds are dissolved in a buffer such as PBS so as to obtain a concentration of 10 to 1,000 mmol/L. The solution of the low molecular compound is brought into contact with the medium having the substrate to which temperature responsive protein A is immobilized to block the unreacted active ester group on the substrate surface. The reaction temperature may be set, for example, within the range of 4 to 37° C. and the reaction time is preferably set within the range of, e.g., 2 minutes to 16 hours.

The medium having the substrate to which temperature responsive protein A is immobilized is stored in a storage solution consisting of a neutral solution within the range of pH4 to 8 at a temperature as low as about 2 to 10° C. As the storage solution, 20% ethanol is preferable in consideration of antibacterial activity.

As described above, temperature responsive protein A binds to an antibody at a low temperature and dissociates from the antibody at a higher temperature than the temperature at which it binds to an antigen. Accordingly, in purifying an antibody by use of a column charged with the medium having temperature responsive protein A, first, a solution containing an antibody as a target to be purified and impurities is poured in the column charged with the medium having temperature responsive protein A under low temperature conditions. The low temperature conditions refer to, for example, 0° C. or more and less than 20° C., preferably 1° C. or more and less than 15° C., and most preferably 2° C. or more and less than 13° C. In this way, the antibody as the target to be purified is trapped by temperature responsive protein A that the medium has. In contrast, since impurities are not trapped by temperature responsive protein A, they flow away together with a solvent from the column.

Note that before the solution containing the antibody as the target to be purified and the impurities is poured in the column charged with the medium having temperature responsive protein A, the solution was roughly purified, for example, by a microfilter having a micropore size of 0.2 μm.

Next, the interior of the column is optionally washed to remove a substance(s) nonspecifically adsorbed to the medium surface. Thereafter, e.g., a buffer is fed to the column under high temperature conditions to dissociate the antibody from protein A that the medium has and a solution containing a purified antibody is collected. The high temperature conditions refer to, for example, 20° C. or more and less than 60° C., preferably 25° C. or more and less than 50° C. and most preferably 30° C. or more and less than 45° C. In this manner, the solution containing the purified antibody from which impurities are removed can be obtained.

Also after the antibody is purified by the temperature responsive protein A medium, the antibody is preferably not to be subjected to a treatment of substantially reducing the affinity for an antigen. The treatment which reduces the affinity for an antigen is not particularly limited; however the aforementioned treatments may be used.

The affinity of the purified human monoclonal antibody for an antigen can be quantified by a known measurement method based on e.g., a dissociation rate constant and a dissociation constant. As the known measurement method, e.g., an enzyme-linked immunosorbent assay (hereinafter referred to as ELISA) and a bio-sensing method (hereinafter referred to as Biacore) using the principle of surface plasmon resonance (Journal of Immunological Method, 145, 229, 1991) are used. In the measurement by Biacore, a minor mass change produced by association and dissociation between two molecules on the surface of a sensor tip is detected based on an optical phenomenon, as a surface plasmon resonance (SPR) signal.

More specifically, an antigen is immobilized to the surface of the sensor tip. Then, the solution containing the purified antibody is continuously supplied to the surface of the sensor tip for a predetermined time by means of a continuous liquid feeding system through a micro flow channel system. A minor mass change is produced in accordance with association and dissociation between the antigen and the antibody on the surface of the sensor tip and detected as the SPR signal.

When an antibody is continuously added at a constant speed for a predetermined time, the antibody is bound to the antigen immobilized to the sensor tip. From this, it is possible to obtain the association rate constant between the antibody and the antigen. Furthermore, the dissociation rate constant between the antibody and the antigen can be obtained by feeding a buffer alone and monitoring the dissociation of the antibody biding to the antigen after addition of the antibody is complicated.

The dissociation constant can be given by the ratio of the dissociation rate constant and the association rate constant, as shown in the following expression (1):

Dissociation constant(K_{D} value)[M]=dissociation rate constant(K_{d} value)[S⁻¹]/association rate constant(Kₐ value)[M⁻¹S⁻¹] (1)

The dissociation constant (K_{D} value) of the antibody purified by the method according to the embodiment relative to an antigen is smaller than the K_{D} value of an antibody purified by an acid elution type protein A medium.

The dissociation rate constant (K_{d} value) of the antibody purified by the temperature responsive protein A medium according to the embodiment relative to an antigen is preferably smaller than the K_{d} value of the antibody purified by the acid elution type protein A medium.

Conventionally, almost all antibodies are purified by acid elution type protein A. In contrast, the present inventors found that the antibody obtained by temperature responsive affinity chromatography is improved in affinity for an antigen. According to the purification method of the aforementioned embodiment based on the finding, a high-affinity human polyclonal antibody can be obtained with high purity and in high yield.

### EXAMPLES

The embodiment will be more specifically described by way of the following Examples; however, the present invention is not particularly limited by the following Examples.

### Example 1

### Preparation of Temperature Responsive Protein a Medium

After a carboxyl group was introduced into crosslinked polyvinyl alcohol beads, the carboxyl group was activated by NHS. Furthermore, the crosslinked polyvinyl alcohol beads activated by NHS were brought into contact with temperature responsive protein A to immobilize temperature responsive protein A to the crosslinked polyvinyl alcohol beads. The procedure is more specifically as follows.

1) Introduction of Carboxyl Group

A reaction solution was prepared by dissolving succinic anhydride (3.0 g) and 4-dimethylaminopyridine (3.6 g) in toluene (450 mL). Then, crosslinked polyvinyl alcohol beads (average particle size 100 μm) (7.5 mL), which were prepared in accordance with the method described in Example 1 of Japanese Patent Laid-Open No. 59-17354, were brought into contact with the reaction solution at 50° C. and stirred for 2 hours. In this manner, a carboxyl group was introduced into the crosslinked polyvinyl alcohol beads. Thereafter, the crosslinked polyvinyl alcohol beads were washed with dehydrated isopropyl alcohol.

2) Activation by NHS

The beads (3 mL) having the carboxyl group introduced therein were added to a NHS activation reaction solution (NHS: 0.09 g, dehydrated isopropyl alcohol: 60 mL, diisopropyl carbodiimide: 0.12 mL) and reacted at 40° C. for 30 minutes. In this manner, the carboxyl group on the beads surface was activated by NHS. After completion of the reaction, the beads were washed with ice cooled dehydrated isopropyl alcohol and further washed with ice-cooled 1 mM hydrochloric acid.

3) Coupling of Temperature Responsive Protein A

Temperature responsive protein A was prepared with reference to Example 11 of Patent Literature (WO2008/143199). A temperature responsive protein A solution was prepared by dissolving temperature responsive protein A (150 mg) in 3 mL of a coupling buffer (0.2 mol/L phosphate buffer, 0.5 mol/L NaCl, pH8.3). Subsequently, the beads activated by NHS were added to the temperature responsive protein A solution. A reaction was carried out at 25° C. while shaking for 4 hours. After a lapse of a predetermine time, the beads were washed with the coupling buffer to wash away temperature responsive protein A not coupled with the NHS activated group on the medium, and collected.

5) Blocking

The beads coupled with temperature responsive protein A were soaked in a blocking reaction solution (0.5 mol/L ethanolamine, 0.5 mol/L NaCl, pH8.0) (10 mL) and allowed to stand still at room temperature for 30 minutes to block the remaining NHS with ethanolamine. After completion of the reaction, the beads were washed with pure water. Thereafter, the beads were enclosed in a column containing 20% ethanol and were stored as it is at 4° C.

(Purification of Antibody by Temperature Responsive Protein a Medium)

A vacant column (GE Health Care Japan Corporation, Tricorn 5/100 column) was charged with the temperature responsive protein A medium by a method in accordance with the instruction provided by the manufacturer. Subsequently, the column was installed in a chromatography system (GE Health Care Japan Corporation, AKTA FPLC).

A culture supernatant containing AE6F4 antibody (0.115 mg/L) as a human monoclonal antibody to which a treatment of reducing the affinity for an antigen was not applied was prepared. AE6F4 production cells were provided by Associate Professor, Yoshinori Katakura of the Agricultural Research Institute, Graduate School of Kyushu University. The AE6F4 antibody production cells were cultured with reference to the literature (Proceedings of the Society for Biotechnology, Japan, Vol. 65, p. 65, 1994). The culture solution containing the AE6F4 antibody production cells was filtered by use of a filtration film (trade name: BioOptimal (registered trade mark) MF-SL, manufactured by Asahi Kasei Medical Co., Ltd.) to remove cells and obtain a solution (culture supernatant) containing the antibody. The filtration was carried out with reference to the instruction provided by the manufacturer.

Subsequently, 6 hours after the solution containing the antibody (culture supernatant) was obtained, the antibody-containing solution was poured in the column charged with the temperature responsive protein A medium under the following conditions to have the medium adsorb the antibody. The temperature of each step was controlled by soaking the column and a pipe (1m) upstream of the column in a constant-temperature vessel set at a predetermined temperature. Furthermore, the column was washed under the following conditions and thereafter, the antibody was eluted from the column (sample A).

1-1) Adsorption Step
Antibody concentration: 0.115 mg/mL
Equilibration buffer: 20 mM phosphate buffer+150 mM NaCl (pH8.0)
Equilibration: 10 beads volume (adsorption buffer was used)
Antibody loading amount: 100 mL
Flow rate: 0.4 mL/min
Beads volume: 1.96 mL
Adsorption temperature: 2° C.

1-2) Washing Step
Washing buffer: the same as adsorption buffer
Flow rate: 0.4 mL/min
Washing temperature: 2° C.

1-3) Elution Step
Elution buffer: the same as adsorption buffer
Flow rate: 0.4 mL/min
Amount of permeate: 20 mL
Elution temperature: 40° C.

### (Concentration Measurement of Antibody and Aggregate)

The concentration of the antibody contained in the elution was obtained by measuring UV absorption at 280 nm and calculating in accordance with the following expression (2).

Antibody concentration(mg/mL)=Absorbance/1.38 (2)

The yield of the antibody was calculated in accordance with the following expression (3).

Yield(%)=100×((amount of antibody collected in washing step)+(amount of antibody collected in elution step))/(amount of antibody loaded in adsorption step). (3)

As the evaluation system of antibody aggregation in the embodiment, a high performance liquid chromatography system was used. More specifically, using the system obtained by connecting a reservoir tank (mobile phase, 0.1 mol/L phosphoric acid, 0.2 mol/L arginine, pH6.8), a liquid feeding pump (feeding liquid linear speed: 1.68 cm/min), a sample loop (volume: 100 μL), a column (room temperature), a detector (UV ray, wavelength: 280 nm) and a drain in the order, a target substance was loaded and then, the absorbance detected by the detector was obtained. The ratio of aggregate contained in the target substance was quantified based on the absorbance. TSKGEL G3000SWXL column manufactured by Tohso Corporation, having an inner diameter (diameter) of 7.8 mm and a bed height of 300 mm was used. Typically, a peak (peak A) of an aggregate containing a dimer or more was detected, until an elution time of 16 minutes, and a monomer peak (peak B) was detected in an elution time of 16 to 18 minutes. From the area ratio of these peaks, degree of antibody aggregation was calculated by use of a program using the following expression (4).

Degree of antibody aggregation(%)=100×(peak A area ratio) (4)

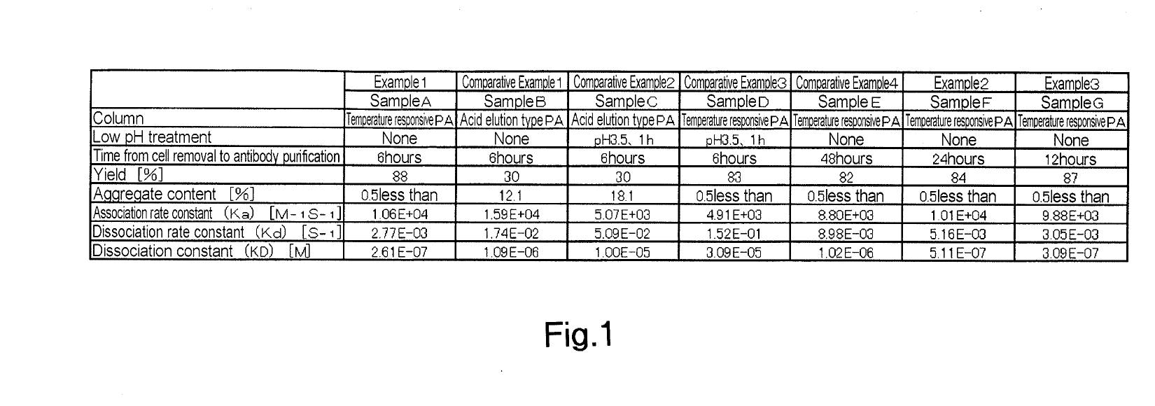

The yield of the antibody was 88%, which was high. Aggregates contained in a fraction of solution eluted from the temperature responsive protein A medium column in the temperature elution step was measured. As a result, it was shown in FIG. 1 that aggregates of the antibody are substantially not contained (less than 0.5%).

The affinity of the antibody for an antigen was measured by Biacore J (registered trade mark) (GE Health Care Japan Corporation). First, to a sensor tip (GE Health Care Japan Corporation, CMS, research grade), cytokeratin 8 (manufactured by PROGEN Biotechnik GmbH), which was an antigen to AE6F4 antibody, was immobilized. As for immobilization method, immobilization was performed by an amine coupling method using an amine coupling kit (catalog No. BR-1000-50, GE Health Care Japan Corporation) in accordance with the instruction provided by the manufacturer. As the running buffer during measurement, HBS-EP buffer (catalog No. BR-1001-88, GE Health Care Japan Corporation) was used as it was and fed at a flow rate of 30 μL/minute at a preset temperature of 25° C. As a pretreatment for the Biacore measurement, the antibody was loaded to a fractionation gel filtration column (GE Health Care Japan Corporation, HiLoad 16/60 Superdex 200 prep grade) to obtain an antibody monomer alone by fractionation. For purification by the fractionation gel filtration column, 50 mM phosphate buffer+150 mM NaCl (pH7.2) was used at a mobile-phase flow rate of 1 mL/minute and in an injection amount of 1 mL. Typically, a monomer peak was detected in an elution time of 60 to 70 minutes. Conditions other than these were set as described in the instruction provided by the manufacturer. The buffer of the antibody purified by the fractionation gel filtration column was replaced with a running buffer used for Biacore measurement. The injection amount of antibody to Biacore was set at 200, 400, 800, 1600, 3200 nM and the dissociation constant was calculated based on the following expression (5) in accordance with the instruction provided by the manufacturer of the apparatus.

Dissociation constant(KD value)[M]=Dissociation rate constant(Kd value)[S⁻¹]/Association rate constant(Ka value)[M⁻¹S⁻¹] (5)

The measurement results by Biacore were summarized in FIG. 1. The dissociation constant (KD value) of the antibody (sample A) purified by a temperature responsive protein A medium column was 2.61×10⁻⁷ [M], which was satisfactory affinity.

### Comparative Example 1

### Purification of Antibody by Acid Elution Type Protein A Medium

The temperature responsive protein A medium was used in Example 1. On the contrary, an acid elution type protein A medium column (MabSelect, GE Health Care Japan Corporation) was used in Comparative Examples 1 and 2. Conditions for antibody adsorption and elution were as follows:

1-1) Adsorption Step
Antibody concentration: 0.115 mg/mL
Equilibration buffer: 20 mM phosphate buffer+150 mM NaCl (pH8.0)
Equilibration: 10 beads volume (adsorption buffer was used)
Antibody loading amount: 100 mL
Flow rate: 0.4 mL/min
Beads volume: 1.96 mL
Adsorption temperature: 25° C.

1-2) Washing Step
Washing buffer: the same as the adsorption buffer
Flow rate: 0.4 mL/min
Washing temperature: 25° C.

1-3) Elution Step
Elution buffer: 50 mM citric acid buffer+0.3M NaCl (pH3.0)
Flow rate: 0.4 mL/min
Amount of permeate: 20 mL
Elution temperature: 40° C.

The antibody as a target to be purified is the same as in Example 1. The hydrogen ion exponent of the recovered eluate as measured by a pH meter was pH3.5. Immediately after elution, 1M tris/hydrochloric acid buffer (pH8.0) was titrated. As a result, the eluate was controlled so as to have a hydrogen ion exponent of pH5.0 and this was specified as sample B.

(Concentration Measurement of Antibody and Aggregate)

The yield of the antibody was 30%, which was low. Aggregates contained in the fraction of the solution (sample B) not treated with acid were measured. As a result, as shown in FIG. 1, antibody aggregates were contained in a large amount of 12.1% (sample B).

The antibody of the above sample B was measured for affinity in the same manner as in Example. The results of measurement by Biacore are summarized in FIG. 1. The dissociation constant (K_{D} value) of sample B was 1.09×10⁻⁶ [M], which was 4 times as large as the dissociation constant (K_{D} value) of the antibody purified by the temperature elution type protein A. It was found that the affinity was low.

### Comparative Example 2

### Acid Treatment for Eluate

The same procedure as in Comparative Example 1 was repeated until elution. The eluate of pH3.5 was maintained at room temperature for one hour. Thereafter, the eluate (pH3.5) was titrated with 1M tris/hydrochloric acid buffer (pH8.0) to be pH5.0 and used as sample C.

(Concentration Measurement of Aggregates)

The aggregates contained in the fraction of the solution (sample C) treated with acid were measured. As a result, as shown in FIG. 1, antibody aggregates were contained in a large amount of 18.1% (sample C).

The antibody of sample C was measured for affinity in the same manner as in Example. The results of measurement by Biacore are summarized in FIG. 1. The dissociation constant (K_{D} value) of sample C was 1.00×10⁻⁵ [M], which was at least 38 times as large as dissociation constant (K_{D} value) of the antibody purified by temperature elution type protein A. It was found that the affinity was low.

### Comparative Example 3

The culture supernatant containing AE6F4 antibody in an amount of 0.115 mg/L was titrated by use of acetic acid to be pH3.5 and maintained at room temperature for one hour. Thereafter, the eluate (pH3.5) was titrated by use of 1M tris/hydrochloric acid buffer (pH8.0) to pH5.0. Subsequently, AE6F4 antibody was purified in the same manner as in Example 1 and specified as sample D.

The antibody of sample D was measured for affinity in the same manner as in Example. The dissociation constant (K_{D} value) of sample D was 3.09×10⁻⁵ [M]. The affinity was found to be smaller than that of the antibody obtained by purifying the culture supernatant not treated with acid by temperature elution type protein A. Other measurement values are shown in FIG. 1.

### Comparative Example 4

AE6F4 production cells were cultured and a culture supernatant was obtained. Forty eight hours later, a solution containing an antibody was poured in a temperature responsive protein A column to have the medium adsorb the antibody. The same procedure as in Example 1 was repeated except the above conditions to purify AE6F4 antibody, which was specified as sample E.

The antibody of sample E was measured for affinity in the same manner as in Example. The dissociation constant (K_{D} value) of sample E was 1.02×10⁻⁶ [M]. The affinity was found to be smaller than that of the antibody purified by temperature elution type protein A immediately after the culture supernatant was obtained. Other measurement values are shown in FIG. 1.

### Example 2

AE6F4 production cells were cultured and a culture supernatant was obtained. Twenty four hours later, a solution containing an antibody was poured in the temperature responsive protein A column to have the medium adsorb the antibody. The same procedure as in Example 1 was repeated except the above conditions to purify AE6F4 antibody, which was specified as sample F.

The antibody of sample F was measured for affinity in the same manner as in Example. The dissociation constant (K_{D} value) of sample F was 5.11×10⁻⁷ [M], which was smaller than the values of Comparative Examples. Other measurement values are shown in FIG. 1.

### Example 3

AE6F4 production cells were cultured and a culture supernatant was obtained. Twelve hours later, a solution containing an antibody was poured in the temperature responsive protein A column to have the medium adsorb the antibody. The same procedure as in Example 1 was repeated except the above conditions to purify AE6F4 antibody, which was specified as sample G.

The antibody of sample G was measured for affinity in the same manner as in Example. The dissociation constant (K_{D} value) of sample G was 3.09×10⁻⁷ [M], which was smaller than the values of Comparative Examples. Other measurement values are shown in FIG. 1.

1. A method for manufacturing a high-affinity antibody comprising:
(A) a step of culturing a cell producing a monoclonal antibody,
(B) a step of removing the cell from a solution containing the cell, and
(C) a step of purifying the monoclonal antibody contained in the solution by a temperature responsive protein A medium,
wherein, before the step (C), none of a low pH treatment, a high-temperature treatment performed at 60° C. or more and a purification treatment by affinity chromatography, are included, and
the solution obtained in the step (B) from which the cell is removed is subjected to the step (C) within 24 hours.

2. The method for manufacturing the high-affinity antibody according to claim 1, wherein a dissociation constant (K_{D} value) of the obtained antibody to an antigen is smaller than the K_{D} value of an antibody obtained by a method for manufacturing an antibody comprising a purification step using an acid elution type protein A medium, which adsorbs a monoclonal antibody at pH4 to 9 and elutes the monoclonal antibody at pH2 to 4, in place of the step (C).

3. The method for manufacturing the high-affinity antibody according to claim 1, wherein the cell is a Chinese hamster ovary cell (CHO cell).

4. The method for manufacturing the high-affinity antibody according to claim 1, wherein the antibody is a human antibody.

5. The method for manufacturing the high-affinity antibody according to claim 1, wherein the dissociation rate constant (K_{d} value) of the obtained antibody to the antigen is smaller than the K_{d} value of an antibody obtained by a method for manufacturing an antibody comprising a purification step using an acid elution type protein A medium, which binds the monoclonal antibody at pH4 to 9 and elutes the monoclonal antibody at pH2 to 4, in place of the step (C).

6. The method for manufacturing the high-affinity antibody according to claim 1, wherein, after the antibody is purified by the temperature responsive protein A medium in the step (C), none of a low pH treatment, a high-temperature treatment performed at 60° C. or more and a purification treatment by affinity chromatography are performed.

7. A high-affinity antibody obtained by a method for manufacturing an antibody comprising:
(A) a step of culturing a cell producing a monoclonal antibody,
(B) a step of removing the cell from a solution containing the cell, and
(C) a step of purifying the monoclonal antibody contained in the solution by a temperature responsive protein A medium,
wherein, before the step (C), none of a low pH treatment, a high-temperature treatment performed at 60° C. or more and a purification treatment by affinity chromatography, are included, and
the solution obtained in the step (B) from which the cell is removed is subjected to the step (C) within 24 hours.

8. The high-affinity antibody according to claim 7, wherein a dissociation constant (K_{D} value) to an antigen is smaller than the K_{D} value of an antibody obtained by a method for manufacturing an antibody comprising a purification step using an acid elution type protein A medium, which binds a monoclonal antibody at pH4 to 9 and elutes the monoclonal antibody at pH2 to 4, in place of the step (C).

9. The high-affinity antibody according to claim 7, wherein the cell is a Chinese hamster ovary cell (CHO cell).

10. The high-affinity antibody according to claim 7, wherein the antibody is a human antibody.

11. The high-affinity antibody according to claim 7, wherein a dissociation rate constant (K_{d} value) to an antigen is smaller than the K_{d} value of an antibody obtained by a method for manufacturing an antibody comprising a purification step using an acid elution type protein A medium, which binds the monoclonal antibody at pH4 to 9 and elutes the monoclonal antibody at pH2 to 4, in place of the step (C).

12. The high-affinity antibody according to claim 7, wherein after the antibody is purified by the temperature responsive protein A medium in the step (C), none of a low pH treatment, a high-temperature treatment performed at 60° C. or more and a purification treatment by affinity chromatography are performed.
